(19) United States
(12) Patent Application Publication
(10) Pub. No.: US 2022/0395269 A1
(43) Pub. Date: Dec. 15, 2022

(54) Arthroscopic Suture Management Device

(71) Applicant: The Regents of the University of Colorado, Denver, CO (US)

(72) Inventors: Omer Mei-Dan, Boulder, CO (US); Jacob Segil, Boulder, CO (US)

(21) Appl. No.: 17/834,678

(22) Filed: Jun. 7, 2022

Related U.S. Application Data
(60) Provisional application No. 63/208,737, filed on Jun. 9, 2021.

(51) Int. Cl. A61B 17/04

(52) U.S. Cl. CPC A61B 17/0469; A61B 2017/0496

(57) ABSTRACT

Embodiments include a suture management device including a disk shaped body that defines a top surface that faces away from a body of a patient when the suture management device is coupled to a sleeve of an arthroscopic cannula inserted into the body and a bottom surface that faces toward the body when the suture management device is coupled to the arthroscopic cannula. The suture management device can include a connector configured to couple the disk shaped body to an arthroscopic cannula, and a set of retention features positioned along the disk shaped body, each retention feature in the set of retention features configured to releasably secure a suture.

### CROSS-REFERENCE TO RELATED APPLICATIONS

This application is a nonprovisional and claims the benefit under 35 U.S.C. 119(e) of U.S. Provisional Patent Application 63/208,737, filed Jun. 9, 2021, the contents of which are incorporated herein by reference as if fully disclosed herein.

### FIELD

The described embodiments relate generally to devices for managing sutures during surgical procedures. More particularly, the present embodiments relate to a suture management device for arthroscopic surgery.

### BACKGROUND

Arthroscopic procedures use a camera that is inserted through an incision to view anatomical structures inside a patient and tools inserted through additional incision sites to perform various treatments and procedures on the patient. Arthroscopic procedures can include using sutures to repair damaged tissue inside the patient. These procedures can include passing sutures between different incision sites. A surgeon may use multiple sutures to treat damaged tissue and, at any given time during the procedure, various ends of the different sutures may be positioned outside different incision sites.

### SUMMARY

Embodiments are directed to a suture management device that includes a disk shaped body defining a top surface that faces away from a body of a patient when the suture management device is coupled to an arthroscopic cannula inserted into the body, and a bottom surface that faces toward the body when the suture management device is coupled to the arthroscopic cannula. The suture management device can include a connector configured to couple the disk shaped body to an arthroscopic cannula and position the disk shaped body below an upper port of the arthroscopic cannula. The suture management device can also include a set of retention features positioned along the disk shaped body, each retention feature in the set of retention features configured to releasably secure a suture.

Embodiments are also directed to a suture organizer that includes a body portion defining a disk shaped structure and a set of retention features positioned along the body portion and configured to releasably retain a suture. The suture organizer can also include a connector attached to the body portion and configured to couple the body portion to a sleeve of an arthroscopic cannula. The body portion can be positioned between an upper port of the arthroscopic cannula and a patient when the sleeve of the arthroscopic cannula is inserted in the patient, and the set of retention features is positioned below an upper surface of the upper port when the body portion is coupled to the arthroscopic cannula.

Embodiments also include a suture management device that includes a disk comprising a flexible material that is configured to at least partially conform to a surface of a body when the suture management device is placed on the body. The disk can define a bottom surface that is oriented toward the body, a top surface that is oriented away from the body, and a central opening that is configured to be positioned around an arthroscopic entry point into the body. The suture management device can also include an adhesive material that is coupled to the bottom surface and configured to secure the disk to the body. The suture management device can include a set of retention features that is coupled to the top surface and positioned about the central opening, each retention feature in the set of retention features configured to releasably secure a suture.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example suture management device being used in an arthroscopic procedure;

FIG. 2 shows an example suture management device being used in an arthroscopic procedure;

FIG. 3 shows an example of multiple suture management devices being used in an arthroscopic procedure;

FIGS. 4A and 4B show perspective views of example suture management devices;

FIG. 5A shows a side view of an example suture management device;

FIG. 5B show a top view of the example suture management device shown in FIG. 5A;

FIG. 6 shows an example suture management device;

FIGS. 7A and 7B show example suture management devices with various examples of suture retention features;

FIG. 8 shows an example suture management device with another example of suture retention features; and

FIG. 9 shows another example suture management device.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments described herein are directed to a suture management device that can be used in arthroscopic surgery, other relatively minimally invasive procedures such as laparoscopic procedures, cardiac procedures, urological procedures, or other surgical procedures. The suture management device can be used to temporarily secure one or more sutures that are being used during these procedures. For example, during an arthroscopic procedure, a portion of a suture may be located in a patient's body and one or more ends of the suture may extend from an entry site, such as an incision into the patient's body. The suture management device can be positioned around the arthroscopic entry point and have one or more retention features which can be used to temporarily secure one or both of the ends of the suture. The retention features can allow a surgeon to quickly secure, release, and identify various sutures that are passed into and out of the patient while the surgeon is performing the arthroscopic procedure. In this regard, free ends (or other portions) of various sutures can be easily secured while a surgeon performs one or more other steps of a procedure, and then retrieved by a surgeon from the retention feature as needed.

In some cases, the suture management device can include one or more identifiers that are used to identify and keep track of different sutures. For example, each retention feature can have a unique identifier such that, when a suture is placed in a specific retention feature, a surgeon can easily identify that specific suture at a later time point during the operation. Some arthroscopic procedures can include using multiple different sutures, each of which have one or more of their ends extending from a patient's body at any given time. Further, different sutures and/or different ends of the sutures may need to be used at different times during an arthroscopic procedure. In some cases, a first end of a suture may be extending from a first entry site on a patient and, after the surgeon uses the suture, that end can be extending from a second entry site on the patient. Further, an end of a suture can be passed into and out of the body multiple times and can also be passed between different entry sites one or more times. As the number of sutures increases, the difficulty of keeping track of the sutures increases. Accordingly, the unique identifiers can be used to uniquely identify each suture and/or keep track of different ends of the same suture. In some cases, multiple suture management devices can be used, where each suture management device is located at a different entry site into the patient. The different suture management devices can have corresponding identifiers, which can be used to keep track of each suture and/or the different ends of the same suture that are extending from entry sites on the patient's body.

In arthroscopic procedures, various instruments, sutures, and other devices are continually passed into and out of various entry sites on the patient. For example, an arthroscopic camera may be inserted through a first entry site, a first instrument may be inserted through a second entry site, and a second instrument may be inserted through a third entry site. The surgeon may need to manipulate the instruments in a precise manner to perform various aspects of the surgery. Accordingly, it can be vital for the surgical field outside the patient to remain free from any structures or devices that would interfere with the surgeon's movement of the instruments.

The suture management device can be configured as a disk or other lower profile shape and positioned around an entry site such that is does not obstruct a surgeon's movements during an arthroscopic procedure. In some cases, the suture management device can attach to a sleeve portion of an arthroscopic cannula that is inserted into an entry site on the patient. The suture management device may be located below an upper port where the arthroscopic instruments are being introduced, and thus remain out of the surgeon's way while he or she is performing the procedure. At the same time, attaching the suture management device to a sleeve of a cannula can allow the retention features to be positioned in an easily accessible location that does not interfere with the surgeon's movements, which may help increase the efficiency, speed, and/or reduce surgical mistakes. In some examples, the suture management device can be a standalone device such as a flexible disk that is positioned around an entry site. These example devices may be placed directly on the user and used with or without an arthroscopic cannula, while still providing the benefits of not obstructing the surgeon's movements and/or providing easy access to the retention features that are used to temporarily secure one or more sutures.

In some cases, the suture management device can be used for non-arthroscopic surgical procedures such as open heart, abdominal, orthopedic or other surgical procedures in which a surgeon directly accesses a surgical site through an incision in a patient. In some examples, the suture management device can have different shapes, such as a partial circular shape and/or include multiple parts. For example, the suture management device can have separate halves that are positioned on different sides of the incision cite. In these examples, the suture management device may include a body portion and one or more retention features, but may not couple to cannula.

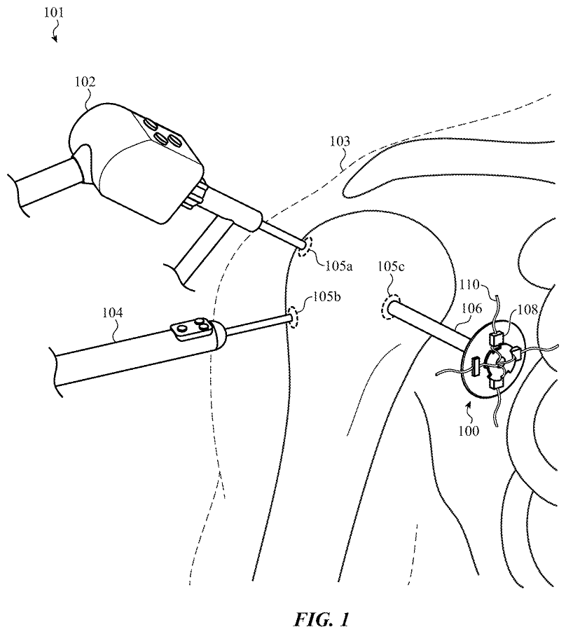

These and other embodiments are discussed below with reference to FIGS. 1-9. However, those skilled in the art will readily appreciate that the detailed description given herein with respect to these Figures is for explanatory purposes only and should not be construed as limiting.

FIG. 1 shows an example suture management device 100 being used in an arthroscopic procedure 101 that is being performed on a patient 103. The arthroscopic procedure 101 can include the use of an arthroscopic camera 102, one or more surgical instruments 104 (one of which is shown for clarity), and the suture management device 100. The arthroscopic camera 102 can be inserted through a first entry site 105a and used to view the surgical site inside the patient 103. The instrument 104 is shown as being inserted into a second entry site 105b, but the instrument 104 can be inserted and removed from any of the entry sites 105. The one or more instruments 104 can include any instruments that are used in arthroscopic surgery or other surgical procedures such as graspers, retrievers, biters, hooks, mallets, punches, and so on. The illustrated arthroscopic procedure 101 shows an example of a shoulder procedure, however, the devices and concepts described herein can be applied to other arthroscopic procedures such as hip, knee, and ankle procedures as well as other minimally invasive surgeries or other surgical operations where suture management would be beneficial.

The suture management device 100 can be coupled to a cannula 106 and have one or more retention features 108 (one of which is labeled for clarity) that are configured to releasably secure one or more sutures 110 (one of which is labeled for clarity). During the arthroscopic procedure 101, the cannula 106 can be inserted into one or more entry sites 105, such as a third entry site 105c shown in FIG. 1. The sutures 110 can be passed through the cannula 106 and out of the patient 103. While the arthroscopic procedure 101 is taking place, ends of various sutures 110 being used in the arthroscopic procedure 101 may be located outside the patient 103. The sutures 110 can be positioned in the retention features 108 while various steps of the arthroscopic procedure are performed. For example, a suture 110 can be secured by a retention feature 108 while the surgeon performs other steps of the procedure that don't involve manipulating the portion of the suture 110 located outside the patient's 103 body. When the suture 110 is needed again, the surgeon can remove it from the retention feature 108 and manipulate it as desired.

One or more instruments 104 can be introduced into the patient 103 through the cannula 106, while the sutures are also extending out of the cannula and secured by the retention features 108. In this regard, a surgeon can insert and remove various instruments 104 through the third entry site 105c in which the suture management device 100 is positioned around. In these cases, the suture management device 100 can secure the sutures 110, while the surgeon performs other portions of the procedure using the third entry site 105c.

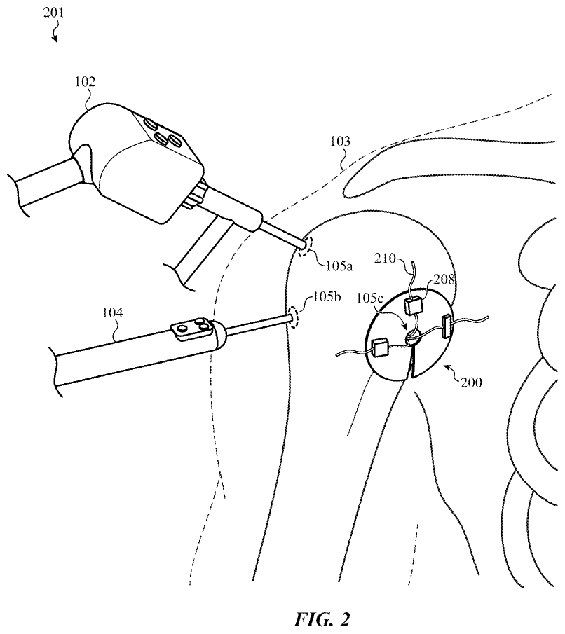

FIG. 2 shows another example suture management device 200 being used in an arthroscopic procedure 201 that is being performed on the patient 103. The arthroscopic procedure 201 can be an example of the arthroscopic procedure described herein and includes the use of various instruments such as the arthroscopic camera 102 and instrument 104, and be performed using entry sites 105.

The suture management device 200 can be placed on the body of the patient 103 and have a central opening that allows access to the third entry site 105c. The suture management device 200 can be flexible and conform to the body surface of the patient 103. In some cases, the suture management device 200 can include one or more retention features 208 (one of which is labeled for clarity) that are configured to releasably secure a suture 210 as described herein. In some embodiments, the suture management device 200 can be a disk that has a bottom surface that prevents it from moving during the arthroscopic procedure. The suture management device 200 can orient the one or more retention features 208 around the third entry site 105c such that the sutures 210 can be secured in a location that reduces interference with introducing other instruments into the third entry site 105c and/or the surgeon's or other health professional's movements while performing the arthroscopic procedure 201. For example, the suture management device 200 can be a thin disk that has retention features positioned along a circular profile and spaced away from the third entry site 105c.

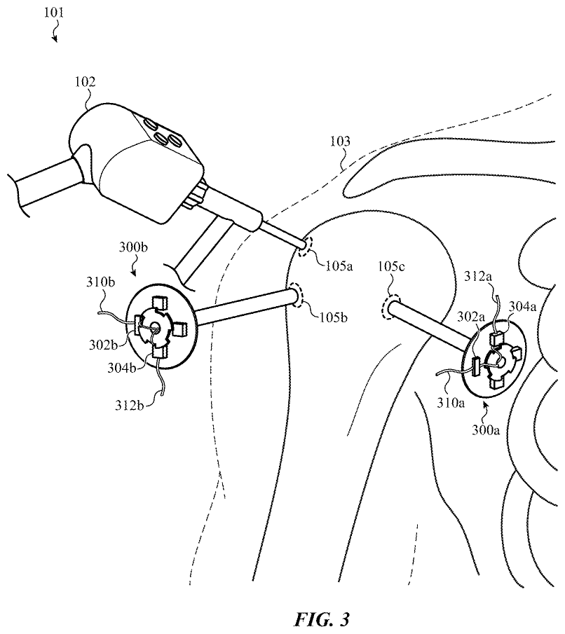

FIG. 3 shows an example of multiple suture managements devices 300 being used in an arthroscopic procedure 301 that is being performed on the patient 103. In some cases, a first suture management device 300a can be positioned at an entry site (e.g., the third entry site 105c) and a second suture management device 300b can be positioned at another one of the entry sites (e.g., the second entry site 105b). The arthroscopic procedure 301 can be an example of the arthroscopic procedures described herein and the suture management devices 300 can be an example of the suture management devices described herein. During the arthroscopic procedure 301, a first suture 310 can be passed through the patient 103, have a first end 310a secured by a first retention feature 302a on the first suture management device 300a and a second end 310b secured by a second retention feature 302b on the second suture management device 300b. In some cases, a second suture 312 can be passed through the patient 103, have a first end 312a secured by a third retention feature 304a on the first suture management device 300a and a second end 312b secured by a fourth retention feature 304b on the second suture management device 300b.

The first and second suture management devices 300 can each have an indicator that uniquely identifies each of the retention features 302 and 304. The indicators can be colors, text, unique shapes of the retention features, other markings, or any other suitable identifier, as described herein. In some cases, an indicator associated with the first retention feature 302a can correspond to an indicator associated with the third retention feature 304a. For example, the first and third retention features 302, 304 can have the same color, marking, shape, or other identifier. Additionally or alternatively, an indicator associated with the second retention feature 302b can correspond to an indicator associated with the fourth retention feature 304b. Accordingly, during the arthroscopic procedure 101, ends of different sutures that extend from different entry sites 105 can readily be identified and associated with each other. For example, the indicators can be used to identify the first end 310a of the first suture 310 at the third entry site 105c and the second end 310b of the first suture 310 at the second entry site 105b. Without such devices, a surgeon or other medical professional may not be able to readily identify which ends correspond to which suture.

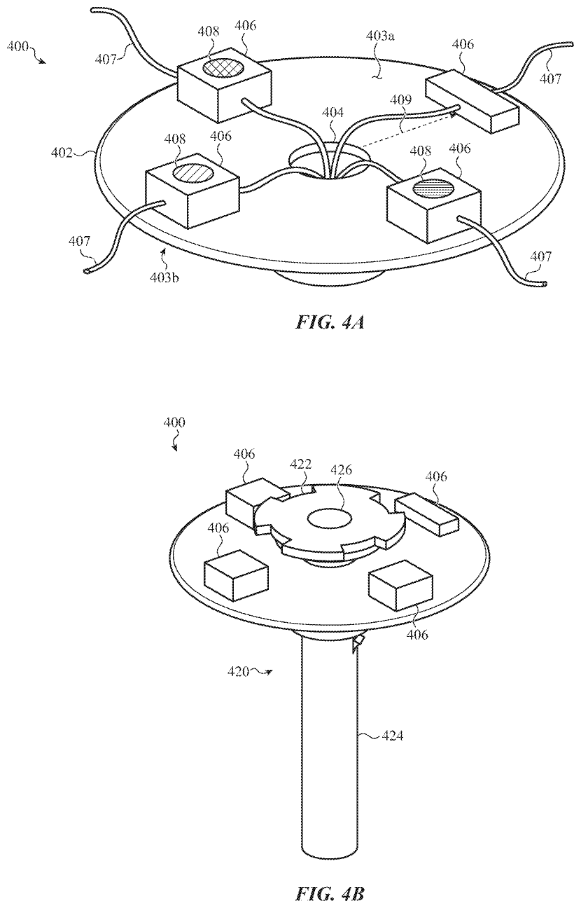

FIG. 4A shows a perspective view of an example suture management device 400. The suture management device 400 can be an example of the suture management devices described herein and include a body portion 402, a connector 404, one or more retention features 406 which can be examples of the retention features described herein, and one or more indicators 408. The suture management device 400 can be configured to couple with an arthroscopic cannula that is inserted into an entry site on a patient during a surgical procedure.

The body portion 402 can include a disk shaped structure that defines a top surface 403a that faces away from the body of a patient and a bottom surface 403b that faces toward the body of a patient when the device is being used in an arthroscopic procedure. The body portion 402 can be configured in a variety of different ways. In some cases, the body portion 402 can define a disk shaped structure, which can have a circular outer profile or any other suitable shape such as elliptical, polygonal, and so on. A dimension of the outer profile of the body portion 402 (e.g., diameter) can be greater than a thickness of the body portion 402. In this regard, the body portion of the suture management device may cover an incision site on a patient. In some cases, the disk shaped structure can have a substantially planar profile. In other cases, the body portion 402 can define a disk shaped structure that is non-planar (or has non-planar portions). For example, the body portion 402 can define a disk shaped structure that has a uniform cross section and is curved toward or away from a patient.

In some embodiments, the body portion 402 can have different cross-sectional profiles and/or define different contours. The disk shaped structure can have a non-uniform cross-section. For example, the body portion 402 can define a structure in which the top surface 403a curves toward the body (e.g., axis of curvature is below the top surface 403a) and the bottom surface 403b is flat. In these, cases the curvature of the top surface 403a, can direct the free end of the sutures 407 toward the patient and away from an entry site, which may help keep the sutures organized and prevent interference with other instruments. In other cases, the body portion 402 can define a shape in which both the top surface 403a and the bottom surface 403b curve way from the body (e.g., axis of curvature is above the top surface 403a). In these cases, the curvature away from a patient may provide an offset between the outer profile of the body portion 402 and a skin surface of the patient, which can provide room from a surgeon to manipulate and/or secure one or more of the sutures 407 to the retention features 406.

The body portion 402 can be formed from a variety of different materials including plastics, metals, composite materials, ceramics, or combinations thereof. In some cases, the body portion 402, the connector 404, and the retention features 406 can all be formed from a single continuous material such as by molding or machining operations. In other cases, the body portion 402 can be a first material and other structures such as the connector 404 and/or the retention features 406 can be a second material.

In some embodiments, the connector 404 couples the body portion to an arthroscopic cannula that is inserted into a patient during an arthroscopic procedure. The connector 404 can be an opening in the body portion 402, such as an opening positioned at the center of the body portion 402. In these examples, the suture management device 400 can be coupled to an arthroscopic cannula by inserting the arthroscopic cannula into the connector 404. In some cases, the connector 404 can have fastening features such as treads, tabs, or any other suitable structure that engages with an arthroscopic cannula to couple the suture management device 400 to the arthroscopic cannula. In other cases, the connector 404 can couple to the arthroscopic cannula using a press fit or friction based connections. The connector 404 can couple the suture management device 400 to an arthroscopic cannula such that the suture management device 400 does not move with respect to the arthroscopic cannula once they are coupled. In other cases, the connector 400 can be configured to allow the suture management device 400 to move with respect to an arthroscopic cannula during a procedure, for example, facilitating rotation of the suture management device 400 with respect to an arthroscopic cannula. In some cases, such as those described herein, the connector 404 can allow the suture management device 400 to be removably attached to an arthroscopic cannula, such that the cannula can be engaged and disengaged with the suture management device 400 by a user such as a medical professional.

In some cases, different suture management devices 400 can have different connectors 404 that couple to different types of arthroscopic cannulas. For example, some connectors 404 can be configured to attach to a first type or brand of arthroscopic cannula and other connectors 404 can be configured to attach to a second type or brand of arthroscopic cannula. Different connectors 404 can have different size, shapes and/or attachment mechanism that are used to couple a corresponding suture management device to a specific arthroscopic cannula (or set of arthroscopic cannulas).

The retention features 406 can be positioned along the body portion 402 of the suture management device 400. In some cases, the retention features 406 are positioned on the top surface 403a of the body portion 402. In other examples, the retention features 406 can be positioned along an outer edge of the body portion 402 or other suitable locations as described herein. Each retention feature 406 can be configured to releasably secure a suture 407 that is extending from an entry site on a patient and through an opening in the body portion 402. In some cases, the retention features 406 are positioned along an outer perimeter of the body portion 402 and can secure the sutures 407 such that a length of each suture extends along a radial direction 409 of the body portion 402. In this regard, the retention features 406 can retain the sutures away from the entry site to reduce interference with other instruments or devices being inserted into and/or taken out of the patient through the opening in the body portion 402. FIG. 4A illustrates a suture management device 400 having four retention features, however, the suture management devices described herein can have more or less retention features.

In some embodiments, the retention features 406 can be configured to secure one or more sutures 407 and maintain tension on the portion of the suture extending from the patient. For example, the retention features 406 can include a locking mechanism that resists movement of the suture with respect to the retention feature 406 once the suture has been engaged with the retention feature 406. In some cases, the retention features 406 can be configured such that a surgeon or other medical professional can secure the secure a suture 407 to the retention features using a specific type of tie off, such as a double loop. In other cases, the retention features 406 can allow the suture to slide back and forth, and another device such as a clip and/or weight can be attached to the suture 407 to maintain tension.

The suture management device 400 can include one or more indicators 408 that are associated with the retention features 406. In some cases, a unique indicator 408 can be associated with each retention feature 406. The indictors 408 can include numbering or lettering that is positioned on the body portion 402 and/or a respective retention feature 406. Additionally or alternatively, the indicators 408 can include other text, symbols, colors, or any other suitable features that uniquely identify a corresponding retention feature 406. In some cases, different retention features 406 can have different shapes, sizes, or other characteristics that serve as a unique indicator 408 for that retention feature 406.

FIG. 4B shows a perspective view of an example suture management device 400 coupled to an arthroscopic cannula 420. In some embodiments, the suture management device 400 can be removably coupled to the arthroscopic cannula 420 in a variety of ways such as those described herein. In other cases, the suture management device 400, or portions thereof, and the arthroscopic cannula 420 can be formed from a single material. For example, the suture management device 400 and arthroscopic cannula can be a single piece of molded or machined polymer material.

The arthroscopic cannula 420 can include an upper port 422 and a sleeve 424. The upper port 422 can define a central opening 426 that is used to insert and/or remove surgical instruments or other devices into and out of the patient when the sleeve 424 is positioned within an entry site (incision) in the patient. The retention features 406 can be positioned around the upper port 422. In some case, the suture management device 400 is coupled to the sleeve 424 of the cannula 420. Additionally or alternatively, the suture management device 400 can be configured to couple to the upper port 422 and/or other portions of the cannula 420.

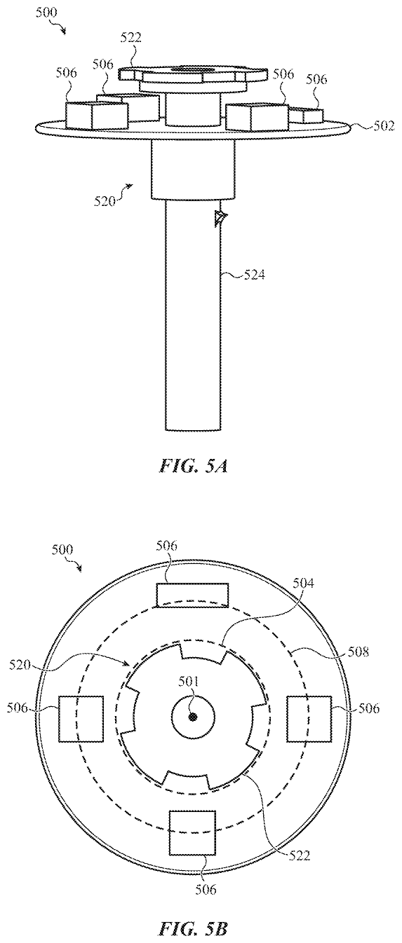

FIG. 5A shows a side view of a suture management device 500 when coupled to the arthroscopic cannula 520. The suture management device 500 can be an example of the suture management devices described herein and include a body portion 502, retention features 506, and be coupled to an arthroscopic cannula 520. The arthroscopic cannula 520 can have an upper port 522 and sleeve 524 that is inserted into a patient during an arthroscopic procedure. In some cases, the retention features 506 can be positioned below an upper surface of the upper port 522. In this regard, when sutures are secured by the retention features 506, they can be positioned below the area where a surgeon is manipulating or otherwise using surgical instruments during an arthroscopic procedure. Accordingly, this configuration of the suture retention features 506 can position the sutures in a location that is easily accessed by the surgeon and/or other medial professional, but also reduces interference with other instruments or actions being performed during the procedure.

FIG. 5B shows a top view of an example suture management device 500 when coupled to the arthroscopic cannula 520. In some cases, one or more of the retention features 506 can be positioned outside an outer profile 504 that is defined by the upper port 522. For example, the retention features 506 can be positioned further from a central axis 501 of the arthroscopic cannula 520 than the outer profile 504 of the upper port 522. Such positioning of the retention features 506 with respect to the upper portion 522 may allow sutures to be easily secured to each of the retention features while reducing their potential for interference with a surgeon's movements and/or other instruments during an arthroscopic procedure. As described herein, the positioning of the retention features 506 with respect to the arthroscopic cannula may help increase efficiency, increase the speed and/or reduce mistakes of an arthroscopic procedure.

In some cases, the retention features 506 can each be positioned the same radial distance from the arthroscopic cannula 520. For example, the retention features 506 can be positioned along circumferential profile 508. Additionally or alternatively, the retention features 506 can be positioned an equal distance from each other. In other cases, the retention features 506 can be positioned at different radial distances from the arthroscopic cannula 520. For example, some retention features can be positioned closer to the arthroscopic cannula 520 than others. In some cases, the distance of the retention features 506 from the arthroscopic cannula 520 can be used as a unique indicator for each retention feature 506. For example, the retention features 506 can be positioned to have a spiral profile, where each retention feature 506 gets progressively further away from the arthroscopic cannula 520.

The size and positioning of the retention features 506 on the body portion 502 can be configured so that a surgeon or other medical professional can easily identify each retention feature and/or an associated indicator, for example, without needing magnification devices. In this regard, the body portion 502 can extend in a radial direction to provide greater surface area for the retention features 506, which can enable advantages such as space for larger and/or more visible retention features, greater spacing between different retention features 506, and/or provide more room for the surgeon or other medical professional to manipulate the sutures. In some cases, greater spacing between different retention features 506 may also reduce mistakes that result from mixing up different sutures due to tight working proximity.

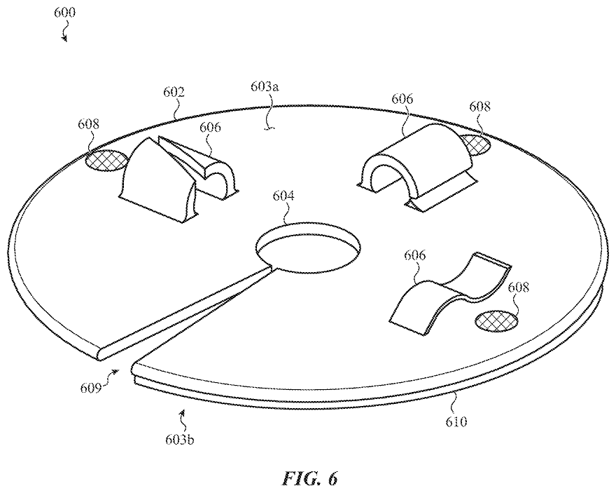

FIG. 6 shows an example suture management device 600 that can be positioned on a surface of a patient. The suture management device 600 can be an example of the suture management devices described herein and can include a body portion 602, an opening 604, and one or more retention features 606. The suture management device 600 can be used with or without an arthroscopic cannula, but may not directly couple to the arthroscopic cannula. For example, the opening 604 can be positioned around an entry site on a patient and be sized to be larger than an outer diameter of a sleeve of an arthroscopic cannula such that the arthroscopic cannula and/or other instruments can be inserted through the opening 604 and into an entry site on a patient.

The body portion 602 can define a disk shaped structure, which can have a circular, elliptical, polygonal, or any other suitable profile. The disk shaped structure can have a profile dimension (e.g., diameter) that is greater than a thickness of the body portion 602. In some cases, the body portion 602 can be a flexible material that at least partially conforms to a surface of a patient. The body portion 602 can define a top surface 603a that is oriented away from a patient's body and a bottom surface 603b that is oriented toward and/or rests on a patient's body. The body portion 602 can be formed from any suitable materials such as polymers, metals, ceramics, composites, or combinations thereof. The body portion 602 and the retention features 606 can be formed from the same or different materials.

In some cases, the body portion 602 can define second opening 609. The second opening 609 can allow the suture management device 600 to be removed from the user while one or more instruments or other devices are inserted in a corresponding entry site. For example, the suture management device 600 can be removed by sliding the instruments through the second opening 609.

The retention features 606 can be examples of the retention features described herein and releasably secure one or more sutures during an arthroscopic procedure. In some cases, each retention feature 606 can be associated with an indicator 608, which can be examples of the indicators described herein. In other cases, the retention features 606 can serve as an indicator. For example, each retention feature 606 can have a unique shape that differentiates it from other retention features 606.

In some cases, the body portion 602 and the retention features 606 are formed from the same material such that the suture management device 600 comprises a single continuous structure. In other cases, the body portion 602 can be formed from a first material and the retention features 606 can be formed from a second material.

The suture management device 600 can also be configured to prevent movement of the body portion with respect to a patient when the device is placed on the patient. In some cases, the body portion 602 can be formed from material(s) that removably adhere to a patient's skin. For example, the body portion 602 can be viscoelastic materials such as hydrogels, or other temporary adhesives. In other cases, the suture management device 600 can include an adhesive material 610 that is coupled to the body portion 602 and secures the suture management device 600 to a patient while a procedure is being performed, and release from the patient once the suture management device 600 is no longer needed. The adhesive material can be any suitable material that releasably secures the suture management device 600 to a patient. In other cases, the suture management device 600 can include features that allow it to be secured to a patient such as by coupling to a drape cloth, having features for suturing the device in place, and so on.

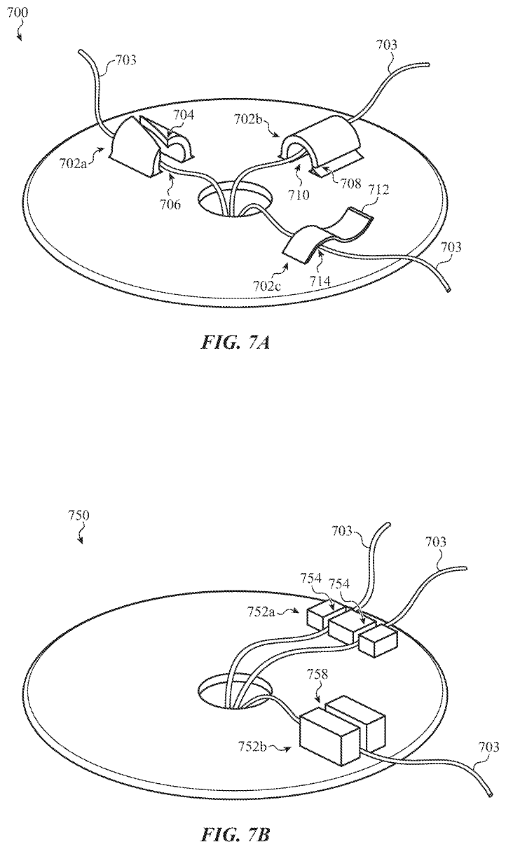

FIG. 7A shows a suture management device 700 with examples of retention features 702 that can be used to secure one or more sutures 703. The suture management device 700 can be an example of the suture management devices described herein and the retention features 702 can be an example of the retention features described herein. The retention features 702 can be implemented in any of the suture management devices described herein and be combined with other examples of the retention features described herein.

A first example retention feature 702a can define a first suture passage 704 and a first internal opening 706. The suture 703 can be passed through the first suture passage 704 and retained within the internal opening 706. In some cases, the first suture passage 704 can be sized to accommodate specific suture sizes. For example, a width of the first suture passage 704 can be substantially similar to or larger than a diameter of the suture 703. In this regard, the first suture passage 704 can be used to selectively accept specific suture sizes. In some cases, the internal opening 706 can be sized to retain one or more sutures 703. Accordingly, the first retention feature 702a can secure multiple different sutures and/or multiple ends of the same suture. Moreover, the first retention feature 702a can retain a suture without pinching or deforming the suture, which may be beneficial to retaining the integrity of the suture.

A second example retention feature 702b can define a second suture passage 708 and a second internal opening 710. The second suture passage 708 can have a ramped/angle feature to help guide the suture 703 into the second internal opening 710. Additionally or alternatively, the second suture passage 708 can have opposite contacting sides that make contact and deform to allow the suture 703 into the internal opening. The second internal opening 710 can be sized to retain one or more sutures.

A third example retention feature 702c can define a third suture passage 712 and a third internal opening 714. The third retention feature 702c can be a clip style feature which, in a first state, the suture passage contacts an upper surface of a body portion and deforms such that the suture 703 can be passed between the body portion and the third retention feature 702c. In some cases, the third retention feature 702a can include a turned up end at the third suture passage 712 that can help guide the suture 703 into the third internal opening 714.

In some embodiments, the retention features 702 can be sized to maintain tension on a portion of the suture the is extending from the patient. For example, the retention features 702 may engage with the suture to prevent it from slipping while engaged with the retention features 702. In this regard, the retention features 702 can be used to tension the suture extending from the patient to keep the suture(s) 703 positioned along an outer portion of the incision site and/or cannula opening.

The retention features 702 described in relation to FIG. 7A are provided as examples, and other features and/or designs are possible.

FIG. 7B shows a suture management device 750 with examples of retention features 752 that can be used to secure one or more sutures 703. The suture management device 750 can be an example of the suture management devices described herein and the retention features 752 can be an example of the retention features described herein. The retention features 752 can be implemented in any of the suture management devices described herein and be combined with other examples of the retention features described herein.

A first example retention feature 752a can include multiple grip portions 754 that are configured to releasably secure one or more sutures 703. In some cases, the grip portions 754 can be configured as slits or narrow passages with a width smaller than a diameter of the suture. In this regard, the grip portions 754 can retain the suture through clamping and/or frictional forces. The second example retention feature 752b includes a single grip portion 758 that is configured to releasably secure one or more sutures 703. The retention features 752 can be formed from a variety of materials such as polymers, metals, ceramics, or other suitable materials. In some cases, the grip portions 754 are configured to maintain tension on a portion of the suture extending from a patient, which can help prevent the sutures from interfering with other instruments being passed into and out of the cannula and/or entry site.

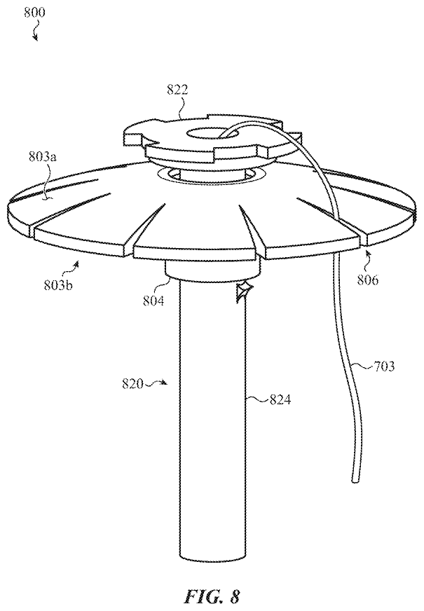

FIG. 8 shows a suture management device 800 with suture retention features 806 (one of which is labeled for clarity) that can be used to secure one or more sutures 703. The suture management device 800 can be an example of the suture management devices described herein. The retention features 806 can be implemented in any of the suture management devices described herein and be combined with other examples of the retention features described herein.

The suture management device 800 can include a body portion 802, which can be an example of the body portions described herein. The suture management device 800 can also include a connector 804 which can be an example of the connectors described herein and be used to couple the suture management device 800 to a sleeve 824 of an arthroscopic cannula 820. In other cases, the suture management device 800 can be formed as part of an arthroscopic cannula such that the suture management device 800 and the arthroscopic cannula form a continuous material structure and/or are otherwise bonded together. The suture management device 800 can be positioned below an upper port 822 of an arthroscopic cannula 820 as described herein, for example, in references to FIGS. 4 and 5. For example, when attached to an arthroscopic cannula that is inserted into a patient, the suture management device 800 can be positioned between a body of a patient and an upper port of the arthroscopic cannular. In some cases, the body portion 802 can have a curved profile that curves toward (as shown in FIG. 8) or away from a patient when attached to an arthroscopic cannula that is inserted into a patient. For example, when the body portion 802 curves toward the patient an axis of the curved profile can be located below the bottom surface 803b. In examples where the body portion 802 curves away from the patient, an axis of the curved profile can be located about the top surface 803a.

The retention features 806 can include one or more openings that extend through the body portion 802 such as from a top surface 803a to a bottom surface 803b of the body portion 802. The retention features 806 can taper inward such that a width of the retention features 806 becomes smaller the closer to the center of the body portion 802 it is. For example, at an outer edge of the body portion 802, the opening of the retention feature is larger than a diameter of the suture 703 and the width of the opening becomes smaller than the suture 703 as it tapers inward. In this regard, the suture 703 can be secured in the retention feature 806 by pulling toward the center of the body portion 802 and removed in an opposite manner. Additionally or alternatively, the retention features can be positioned on the top surface 803a such as the retention features described herein (e.g., retention features 406, 506, 606, 702, and/or 752).

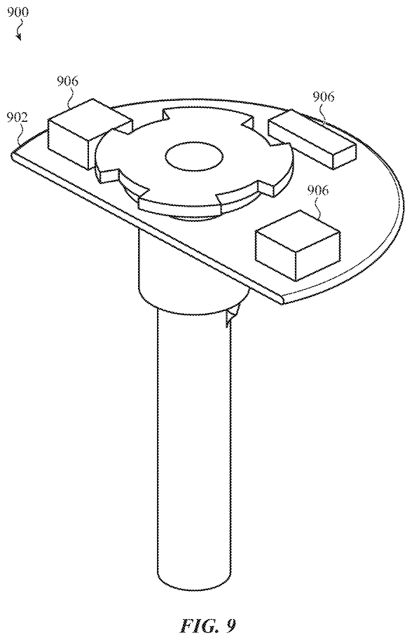

FIG. 9 shows an example suture management device 900 having a body portion 902 that is shaped to reduce interference with other instruments being used in an arthroscopic procedure. For example, the body portion 902 can define a partial disk structure, which can allow multiple suture management device 900 to be used in close proximity and/or leave an opening for accessing other entry sites that are close to the entry site that contains the suture management device 900. In some cases, the body portion 902 can define multiple opening or cutouts. For example, the body portion 902 can define a disk shaped structure where the distance between the periphery and the center of the body portions varies based on the angular position.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A suture management device comprising:
a disk shaped structure defining:
a top surface that faces away from a body of a patient when the suture management device is coupled to an arthroscopic cannula inserted into the body; and
a bottom surface that faces toward the body when the suture management device is coupled to the arthroscopic cannula;
a connector configured to couple the disk shaped body to a sleeve of the arthroscopic cannula; and
retention features positioned along the disk shaped body, each retention feature releasably securing a suture.

2. The suture management device of claim 1, wherein the connector defines an opening that positioned over the sleeve of the arthroscopic cannula.

3. The suture management device of claim 1, wherein:
the disk shaped body is between an upper port of the arthroscopic cannula and the body; and
each retention feature is positioned further from a central axis of the arthroscopic cannula than an outer profile of the upper port of the arthroscopic cannula when the suture management device is connected to the arthroscopic cannula.

4. The suture management device of claim 1, wherein:
each retention feature defines:
a suture passage; and
an internal opening; and
the internal opening retains a respective suture that is passed through the suture passage.

5. The suture management device of claim 1, wherein:
each retention feature is positioned on the top surface; and
each retention feature positions a length of the suture along a radial direction of the disk shaped body.

6. The suture management device of claim 1, wherein:
each retention feature defines an opening extending from the top surface to the bottom surface; and
a first portion of the opening that is positioned further from a center of the disk shaped body has a first width greater than a second portion of the opening that is positioned closer to the center of the disk shaped body.

7. The suture management device of claim 6, wherein:
the disk shaped body defines a curved profile; and
an axis of the curved profile is below the top surface.

8. The suture management device of claim 1, wherein the disk shaped body has a circular outer profile.

9. A suture organizer, comprising:
a body portion defining a disk shaped structure;
retention features positioned along the body portion and configured to releasably retain a suture; and
a connector attached to the body portion and configured to couple the body portion to a sleeve of an arthroscopic cannula; wherein:
the body portion is positioned between an upper port of the arthroscopic cannula and a patient when the sleeve of the arthroscopic cannula is inserted in the patient; and
the set of retention features is positioned below an upper surface of the upper port when the body portion is coupled to the arthroscopic cannula.

10. The suture organizer of claim 9, wherein:
the disk shaped structure defines a circular outer profile; and
each retention feature is positioned on a top surface of the body portion.

11. The suture organizer of claim 10, wherein each retention feature is positioned outside an outer profile of the upper port when the suture organizer is coupled to the arthroscopic cannula.

12. The suture organizer of claim 11, wherein each retention feature positions a length of a respective suture along a radial direction of the disk shaped structure.

13. The suture organizer of claim 9, wherein:
the suture organizer comprises a set of indicators; and
each indicator in the set of indicators is associated with a retention feature in the set of retention features.

14. The suture organizer of claim 13, wherein each indicator in the set of indicators uniquely identifies a respective retention feature.

15. The suture organizer of claim 9, wherein the body portion is formed from a flexible material and configured to at least partially conform to an underlying surface of the patient.

16. The suture organizer of claim 9, wherein the body portion and the connector are formed from a single continuous material structure.

17. A suture management device, comprising:
a disk comprising a flexible material that:
at least partially conforms to a surface of a body when the suture management device is placed on the body; and
prevents movement of the disk with respect to the body, the disk defining:
a bottom surface that contacts the body;
a top surface that is oriented away from the body; and
a central opening that is configured to be positioned around an arthroscopic entry point into the body; and
a set of retention features coupled to the top surface and positioned about the central opening, each retention feature in the set of retention features configured to releasably secure a suture.

18. The suture management device of claim 17, further comprising a set of indicators, wherein each indicator is associated with a different retention feature in the set of retention features.

19. The suture management device of claim 17, wherein the set of retention features are formed from the flexible material.

20. The suture management device of claim 19, wherein:
each retention feature in the set of retention features comprises an internal opening; and
the respective suture is positioned within the internal opening to secure the respective suture to the suture management device.
